(19) United States
(12) Patent Application Publication
(10) Pub. No.: US 2015/0202124 A1
(43) Pub. Date: Jul. 23, 2015

(54) Exfoliating Body Soap

(71) Applicant: Maureen E. Monteiro, Chevy Chase, MD (US)

(72) Inventor: Maureen E. Monteiro, Chevy Chase, MD (US)

(21) Appl. No.: 14/599,862

(22) Filed: Jan. 19, 2015

Related U.S. Application Data
(60) Provisional application No. 61/929,301, filed on Jan. 20, 2014.

(51) Int. Cl. A61K 8/02; A61Q 19/10; A61K 8/97; A61K 8/34; A61K 33/06; A61K 8/92

(52) U.S. Cl. CPC A61K 8/0216; A61K 33/06; A61K 8/922; A61K 8/97; A61K 8/34; A61Q 19/10

(57) ABSTRACT

Exfoliating body soap cleanses and removes oils and particulates from the user's skin. The exfoliating body soap includes a cleansing surfactant base, a first quantity of exfoliating agent, and a second quantity of exfoliating agent. The cleansing surfactant base emulsifies and binds water insoluble oils and particulates present on a user's skin to be washed away using water. The first quantity of exfoliating agent and the second quantity of exfoliating agent are an abrasive material which exfoliates the user's skin, removing dead skin while exposing newer layers of skin. The first quantity of exfoliating agent is dispersed throughout the cleansing surfactant base. The second quantity of exfoliating agent is concentrated on an external surface of the cleansing surfactant base. This arrangement allows the user to have a consistent abrasiveness throughout the exfoliating body soap and a concentrated portion which the user can focus the abrasiveness of the exfoliating body soap.

The current application claims a priority to the U.S. Provisional Patent application Ser. No. 61/929,301 filed on Jan. 20, 2014.

### FIELD OF THE INVENTION

The present invention relates generally to soap soap. More specifically, the present invention is made from composite ingredients in order to exfoliate, cleanse and relax through the skin of the user when properly applied.

### BACKGROUND OF THE INVENTION

The skin is the protective coating that envelopes the human's whole body and is responsible for the protection of the organs inside. Skin also regulates body temperature through sweat glands and works in conjunction with the nervous system to alert the body of various environmental conditions. Skin further creates and absorbs essential vitamins and minerals necessary for the body's survival from environmental and topical elements. Skin consists of three different layers: the epidermis, the dermis and the subcutaneous layer. The epidermis portion that is exposed to the environment comprises dead skin cells that are constantly being shed and replaced by newer skin cells. These new cells produced in the lower layers of the epidermis every 28 days. Proper hygiene allows the skin to function optimally. Dirt and dust settles and adheres to the skin adversely affecting the temperature regulating properties of skin. To remove such dust and dirt, washing and exfoliating the epidermal layer on a regular basis is required. Soap is typically chosen to clean the epidermal layer. Most bars of soap comprise natural oils or fats that have been treated with sodium hydroxide or other strong alkali. During the cleaning process, soap acts as an emulsifying agent and traps insoluble particles resulting in a water soluble micelle which can be washed away simply with water. Soap has been known to be used since the ancient Babylon civilization where alkali and cassia oil was used instead. Since then, soap has evolved to embody a variety of different designs and compositions. Through the use of soap, a user washes the outer layer removing dirt, debris, and other small particles from the skin; it does not remove old dead skin cells and an excess of dead cells on the outer layer of the skin clogs pores, produces acne, and traps dirt. Exfoliating once or twice a week sheds these unwanted dead cells from the skin and produces a healthy growing environment for new born skin cells. This can be achieved through chemical or mechanical means. Chemical exfoliates utilize acidic properties of various compounds to weaken the cohesive properties of the skin cells in order to remove them. Mechanical means, on the other hand, utilize physical agitation to manually scrub dead skins off the top epidermal layer.

The majority of the population only washes their skin with soap, they do not exfoliate. The importance of exfoliation is not well known in the modern community due to lack of awareness and access to facilities. The present invention seeks to provide a product which contains both exfoliation qualities and soap qualities. The combining of the two features encourages proper skin hygiene in a safe, convenient, and simple manner. Soaking in warm or hot water causes the pores to expand. Toxins are released from the body through the pores while minerals are received through the pores to enhance the relaxing effect. Using the Exfoliating Body Soap to bathe and/or soak offers a refreshing feeling, cleansing toxins, and removing particulates from the body of the user.

### BRIEF DESCRIPTION OF THE DRAWINGS

FIG. 1 is a schematic view of the present invention.

FIG. 2 is a block diagram illustrating the active ingredients of the cleansing surfactant base.

FIG. 3 is a block diagram illustrating the ingredients of the first quantity of exfoliating agent and the second quantity of exfoliating agent.

FIG. 4 is a block diagram illustrating the ingredients of the strong alkali.

FIG. 5 is a flow diagram illustrating the method of manufacture of the present invention.

FIG. 6 is a flow diagram illustrating two different processes for adding the second quantity of exfoliating agent to the preparatory mixture.

### DETAIL DESCRIPTIONS OF THE INVENTION

All illustrations of the drawings are for the purpose of describing selected versions of the present invention and are not intended to limit the scope of the present invention.

The present invention is for exfoliating body soap which removes small particles and dead skin cells from the outer layer of the skin of a user. Abrasive materials are dispersed throughout a cleaning base such that the present invention is able to effectively both clean and exfoliate the user's skin. The cleansing base emulsifies dust and small particulates such that the user is able to use water to wash away water insoluble compounds present on their skin.

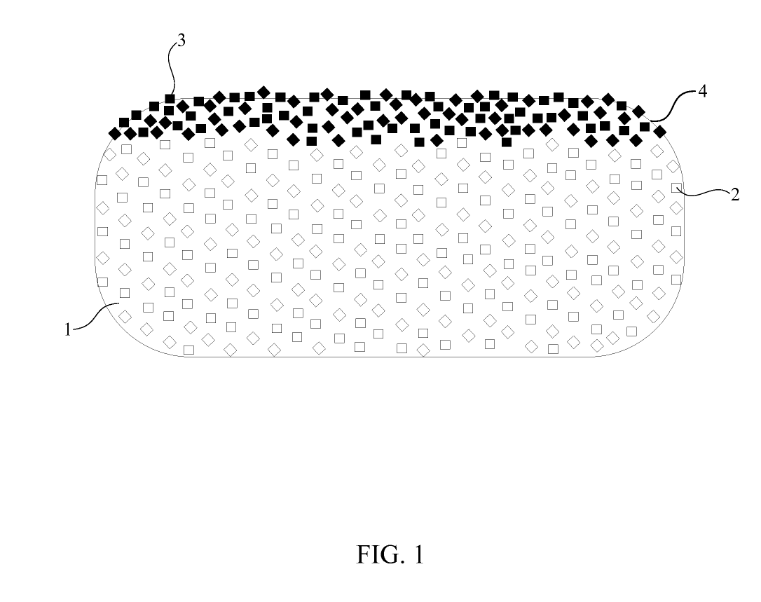

In accordance with FIG. 1, the present invention comprises a cleansing surfactant base 1, a first quantity of exfoliating agent 2, and a second quantity of exfoliating agent 3. The cleansing surfactant base 1 provides a compound that suspends the first quantity of exfoliating agent 2 and the second quantity of exfoliating agent 3, as well as cleans and emulsifies dust and other particulates to be removed from the user's skin through additional use of water. The first quantity of exfoliating agent 2 and the second quantity of exfoliating agent 3 is an abrasive material which removes dead skin from the user. The first quantity of exfoliating agent 2 is dispersed throughout the cleansing surfactant base 1. As the cleansing surfactant base 1 is being dissolved though use, more of the first quantity of exfoliating agent 2 is exposed over time maintaining a relatively constant abrasiveness of slight variance. The second quantity of exfoliating agent 3 is concentrated on an external surface 4 of the cleansing surfactant base 1, which allows the user to target and exfoliate specific sections of their skin. In the preferred embodiment, the cleansing surfactant base 1 is approximately 75% by volume of the present invention and the first quantity of exfoliating agent 2 and the second quantity of exfoliating agent 3 encompass the remainder of the present invention, approximately 25% by volume of the present invention.

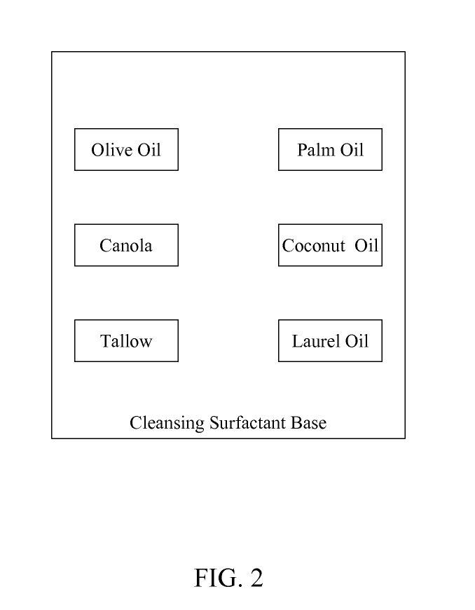

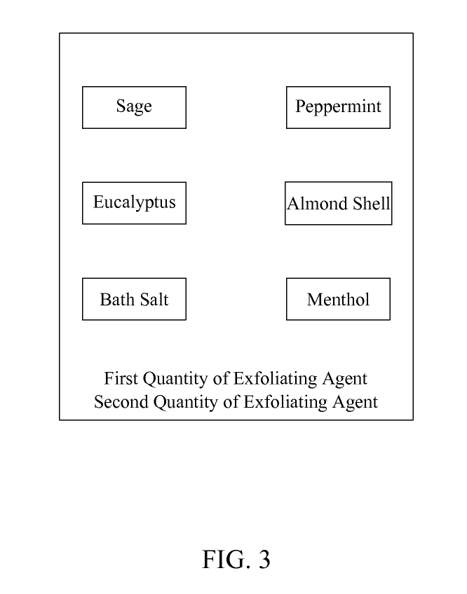

The cleansing surfactant base 1 contains active ingredients selected from a group consisting of palm, coconut, olive, canola, tallow, laurel oils, and combinations thereof, as detailed in FIG. 2. These compounds have been proven effective for cleansing the epidermal layer and formulating a solid soap base. As shown in FIG. 3, the first quantity of exfoliating agent 2 and the second quantity of exfoliating agent 3 are selected from a group consisting of sage, eucalyptus, peppermint, bath salt, almond shell, menthol, and combinations thereof. The compounds for the first quantity of exfoliating agent 2 and the second quantity of exfoliating agent 3 create an abrasive texture. The abrasive texture is formulated by grinding these compounds into particulates and chosen from fine, medium, or course grades, average particle sizes between 68 to 140, 190 to 265 and 336 to 425 micrometers, respectively. The abrasive texture to be incorporated within the present invention for different grits is dependant on various skin types and user preference. Further, these particular ingredients provide the skin with vitamins, minerals, and similar nutritional resources as well as various health benefits. Sage is a natural disinfectant and deodorizer with healing capabilities. Eucalyptus oils treat acne, wounds, inflammations, burns, infections, and joint pains. Peppermint contains ingredients that activate cold-sensitive receptors in the skin and create a cooling sensation. Almond shells, extracts, and oils provide the skin with riboflavin, niacin, thiamin, pantothenic acid, vitamin B-6, folates, and a plurality of minerals. Bath salts are water soluble crystalline-type particles containing natural ingredients and nutrients that create the abrasive texture of the exfoliating portion of the present invention. Some bath salts are energizing and others are soothing. Menthol is an organic compound with local anesthetic, counterirritant, and soothing qualities, ideal for a recently exfoliated epidermal layer.

In some embodiments, the present invention further comprises a muscle relaxing agent. The muscle relaxing agent is topically applied to the user to relax their muscles such that the user's muscles are able to be easily massaged, reducing pain and relaxing the person being massaged. The muscle relaxing agent is homogeneously mixed with the cleansing surfactant base 1, such that the muscle relaxing agent is consistently applied through every used of the present invention. Magnesium is preferred to be the muscle relaxing agent since it is able to be topically absorbed through the skin, but any other topically applied muscle relaxing agent may be used.

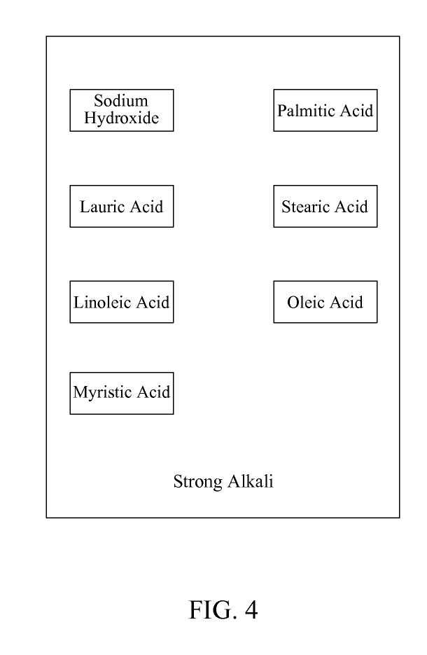

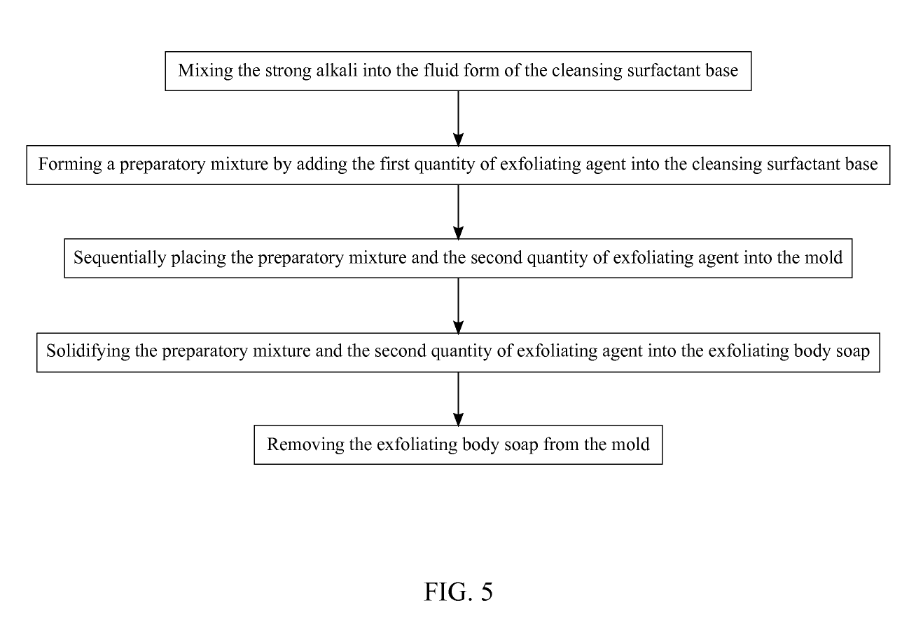

In order to manufacture the present invention detailed in FIG. 5, a user first mixes a strong alkali into a fluid form of the cleansing surfactant base 1. The strong alkali reacts with the oils and fatty active ingredients within the cleansing surfactant base 1, beginning the solidification of the present invention over a period of time. In accordance to FIG. 4, the strong alkali is selected from a group consisting of sodium hydroxide, lauric acid, myristic acid, palmitic acid, stearic acid, oleic acid, linoleic acid, and combinations thereof. These compounds have been found effecting in forming a water soluble micelle which binds and removes particulates from the user's skin. As the exfoliating body soap begins the solidifying reaction, the first quantity of exfoliating agent 2 is added and homogeneously mixed with the cleansing surfactant base 1 forming a preparatory mixture.

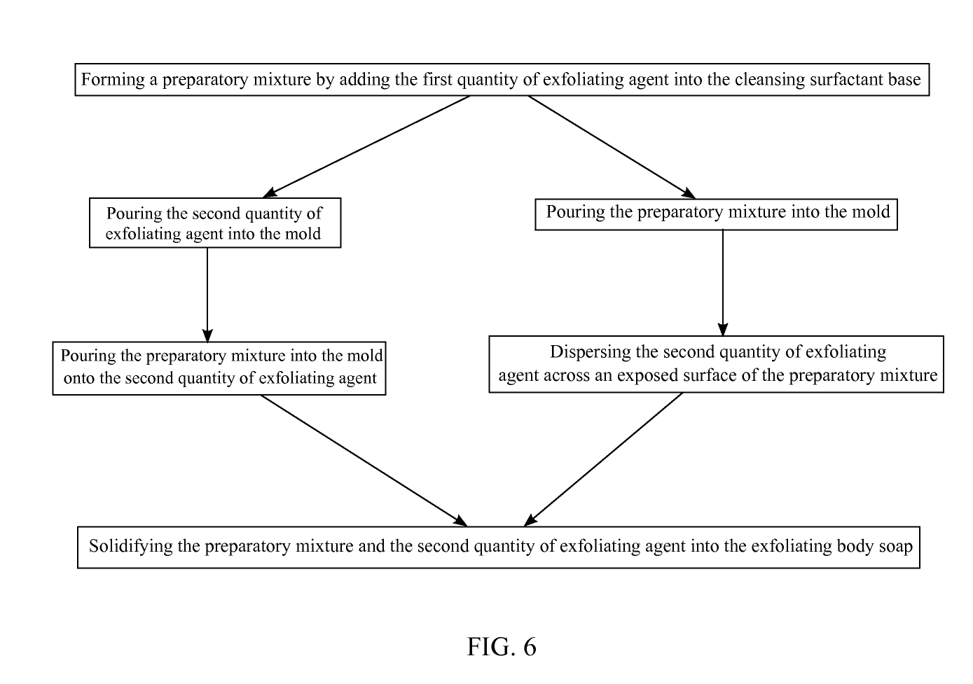

The method of manufacture has two different processes for placing the second quantity of exfoliating agent 3 and the preparatory mixture into a mold, while the preparatory mixture is still in a liquid form, in accordance to FIG. 6. The mold is an impression of the final form of the exfoliating body soap. In one process, the second quantity of exfoliating agent 3 is poured into the mold and the preparatory mixture is poured into the mold onto the second quantity of exfoliating agent 3. In this process, the second quantity of exfoliating agent 3 is dispersed on the external surface 4 of the solid cleansing surfactant base 1 in the form of the mold. In the other process, the preparatory mixture is poured into the mold and the second quantity of exfoliating agent 3 is dispersed across an exposed external surface 4 of the preparatory mixture within the mold. In this alternate process, the second quantity of exfoliating agent 3 is generally dispersed across a level surface. Once the second quantity of exfoliating agent 3 and the preparatory mixture are present within the mold, the preparatory mixture is solidified through a drying process into the exfoliating body soap. Finally, when the exfoliating body soap is solidified, the exfoliating body soap is removed from the mold and ready for packaging or direct use.

Although the invention has been explained in relation to its preferred embodiment, it is to be understood that many other possible modifications and variations can be made without departing from the spirit and scope of the invention as hereinafter claimed.

What is claimed is:

1. An exfoliating body soap comprises:
a cleansing surfactant base;
a first quantity of exfoliating agent;
a second quantity of exfoliating agent;
the first quantity of exfoliating agent being dispersed throughout the cleansing surfactant base;
the second quantity of exfoliating agent being concentrated on an external surface of the cleansing surfactant base; and
the first quantity of exfoliating agent and the second quantity of exfoliating agent being an abrasive material.

2. The exfoliating body soap as claimed in claim 1 wherein, the cleansing surfactant base contains active ingredients selected from a group consisting of palm oil, coconut oil, olive oil, canola, tallow, laurel oil, and combinations thereof.

3. The exfoliating body soap as claimed in claim 1 wherein, the first quantity of exfoliating agent and the second quantity of exfoliating agent are selected from a group consisting of sage, eucalyptus, peppermint, bath salt, almond shell, menthol, and combinations thereof.

4. The exfoliating body soap, as claimed in claim 1, wherein the abrasive material is fine grade particulate.

5. The exfoliating body soap, as claimed in claim 1, wherein the abrasive material is medium grade particulate.

6. The exfoliating body soap, as claimed in claim 1, wherein the abrasive material is course grade particulate.

7. The exfoliating body soap as claimed in claim 1 comprises:
the cleansing surfactant portion being approximately 75% by volume of the exfoliation body soap; and
the first quantity of exfoliating agent and the second quantity of exfoliating agent being approximately 25% by volume of the exfoliation body soap.

8. The exfoliating body soap as claimed in claim 1 comprises:
a muscle relaxing agent; and
the muscle relaxing agent being homogeneously mixed with the cleansing surfactant portion.

9. The exfoliating body soap, as claimed in claim 5, wherein the muscle relaxing agent is magnesium.

10. A method of manufacturing the exfoliating body soap, as claimed in claim 1 comprises the steps of:
providing a mold and a strong alkali;
providing the cleansing surfactant base is a fluid form;
mixing the strong alkali into the fluid form of the cleansing surfactant base;
forming a preparatory mixture by adding the first quantity of exfoliating agent into the cleansing surfactant base;
sequentially placing the preparatory mixture and the second quantity of exfoliating agent into the mold;
solidifying the preparatory mixture and the second quantity of exfoliating agent into the exfoliating body soap; and
removing the exfoliating body soap from the mold.

11. The method of manufacturing and use of a exfoliating body soap, as claimed in claim 9 wherein the strong alkali is selected from a group consisting of sodium hydroxide, lauric acid, myristic acid, palmitic acid, stearic acid, oleic acid, linoleic acid, and combinations thereof.

12. The method of manufacturing the exfoliating body soap, as claimed in claim 11 comprises the steps of:
pouring the second quantity of exfoliating agent into the mold; and
pouring the preparatory mixture into the mold and onto the second quantity of exfoliating agent.

13. The method of manufacturing the exfoliating body soap, as claimed in claim 11 comprises the steps of:
pouring the preparatory mixture into the mold; and
dispersing the second quantity of exfoliating agent across an exposed surface of the preparatory mixture.
